(19) United States
(12) Patent Application Publication
(10) Pub. No.: US 2023/0149483 A1
(43) Pub. Date: May 18, 2023

(54) SHORT-CHAIN FATTY ACID USE TO MITIGATE ANTIMICROBIAL RESISTANCE AND VIRULENCE GENE TRANSFER IN THE GUT

(71) Applicant: Iowa State University Research Foundation, Inc., Ames, IA (US)

(72) Inventors: Melha Mellata, Ames, IA (US); Logan C. Ott, Ames, IA (US)

(21) Appl. No.: 18/055,050

(22) Filed: Nov. 14, 2022

Related U.S. Application Data
(60) Provisional application No. 63/264,010, filed on Nov. 12, 2021.
(60) Provisional application No. 63/268,162, filed on Feb. 17, 2022.

(51) Int. Cl. A61K 35/747; A61K 47/26; A61P 31/04

(52) U.S. Cl. CPC A61K 35/747; A61K 47/26; A61P 31/04

(57) ABSTRACT

The disclosure relates to compositions for inhibiting bacterial conjugation or horizontal plasmid-associated gene transfer. The compositions may comprise short chain fatty acids. The compositions may also comprise short chain fatty acid-producing probiotic bacteria. Also disclosed herein are methods of inhibiting bacterial conjugation or horizontal gene transfer in the gut of a subject.

### CROSS REFERENCE TO RELATED APPLICATIONS

This application claims priority to provisional patent applications U.S. Ser. No. 63/264,010, filed Nov. 12, 2021, and U.S. Ser. No. 63/268,162, filed Feb. 17, 2022, which are incorporated herein by reference in their entireties.

### TECHNICAL FIELD

The present disclosure relates to methods of inhibiting horizontal plasmid gene transfer through bacterial conjugation to control the spread of virulence and antibiotic resistance genes in the gut environment.

### BACKGROUND

The spread of antibiotic resistance genes and the emergence of multidrug resistant bacteria cause clinical failure of antibiotic treatments, thus threatening animal and public health. An estimated 2.8 million antibiotic-resistant infections occur in the U.S. each year, and more than 35,000 people die. Thus, there is a growing need to address the problem of antibiotic resistance. The gastrointestinal tract of animals and humans harbors a vast number of microorganisms that could carry and transfer antibiotic resistance genes. The primary means for the spread of antibiotic resistance is by horizontal gene transfer, and conjugative plasmids are the principal vectors for the horizontal gene transfer of virulence and antibiotic resistance genes, leading to the rapid rise of antibiotic resistance in both pathogenic and commensal bacteria. Failing to tackle multidrug resistance could result in 11 to 444 million deaths globally, and alternative treatments that do not rely on antibiotics are urgently needed.

### SUMMARY

Methods of inhibiting bacterial conjugation or horizontal gene transfer in the gut of a subject are provided. In certain embodiments, the methods comprise administering to the subject a composition comprising a probiotic bacterium and a prebiotic, wherein the probiotic bacterium produces at least one short chain fatty acid (SCFA) in the gut of the subject. In certain embodiments, the methods comprise administering to the subject a composition comprising at least one SCFA.

Compositions for inhibiting bacterial conjugation or horizontal gene transfer in the gut of a subject comprising at least one SCFA and/or a SCFA-producing probiotic bacterium are also provided.

While multiple embodiments are disclosed, still other embodiments of the disclosure will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIG. 1A-B shows in vivo detection of the transfer of antibiotic-resistant plasmids between bacteria in the guts of male and female W¹¹¹⁸ fruit flies. Two conjugative plasmids from the broad-host-range IncP (pKJK-5), and narrow-host-range IncF (pCVM29188_146) plasmid groups were tested for their transferability from a donor E. coli bacteria to a plasmid less recipient E. coli. Log₁₀ CFU/gut transconjugants (FIG. 1A) and conjugation frequency (FIG. 1B, Transconjugants/Donors) are reported. ND, not detected.

FIG. 2A-B shows the in vitro inhibition effect of propionic acid on the transfer of a conjugative plasmid of interest to the poultry industry, pCVM29188_146 (IncF), between a donor E. coli bacterium to a plasmid less recipient E. coli. Log₁₀ CFU/gut (FIG. 2A) and conjugation frequency (FIG. 2B) reported. ND, not detected; *, P-value <0.05; **, P-value <0.005; ****, P-value <0.00005.

FIG. 3 shows the in vitro inhibition effect of SCFAs on the transfer of two conjugative plasmids of interest to the poultry industry, pCVM29188_146 (IncF, Left) and plasmid pAPEC-02-211A-ColV (IncF, Right). Transfer was shown between a donor E. coli bacterium and a plasmid less recipient E. coli, and conjugation frequency (Transconjugants/Donors) were reported. ND, not detected; *, P-value <0.05; **, P-value <0.005; ***, P-value <0.0005; ****, P-value <0.00005.

FIG. 4 shows the in vitro inhibition effect of eight distinct SCFAs on the transfer of the conjugative poultry plasmid pAPEC-02-211A-ColV (IncF) at varying molar concentrations between 0 and 1 molar between a donor E. coli bacterium and a plasmid less recipient E. coli. Log₁₀ CFU/gut Transconjugants is reported. The absence of bars represents the absence of detectable transconjugants. *, P-value <0.05; **, P-value <0.005; ***, P-value <0.0005; ****, P-value <0.00005.

FIG. 5 shows the ex vivo inhibition effect of SCFAs on transfer of the conjugative plasmid pAPEC-02-211A-ColV (IncF) between a donor E. coli bacterium and a plasmid less recipient E. coli using chicken ceca explants. Log₁₀ CFU/gut donors (Top), Recipients (Middle) and Transconjugants (Bottom) were reported. ****, P-value <0.00005.

FIG. 6A-C shows in vitro inhibition of bacterial plasmids conjugation of various plasmid incompatibility types. Log₁₀ CFU/mL of donors (left), Recipients (middle) and transconjugants (right) involved in the transfer of plasmids of the incompatibility types IncP1ε (FIG. 6A), IncFIIβ (FIG. 6B), and IncI1 (FIG. 6C) exposed to water, and 0.025 M acetate, propionate, or butyrate. *, P-value <0.05 with each additional * indicating an order of magnitude increase in significance. Error bars represent the standard error around the mean value of three separate replicates.

### DETAILED DESCRIPTION

Short chain fatty acids (SCFAs) are produced by beneficial gut bacteria when they ferment dietary fiber in the gut of animals and humans. SCFAs are used for energy and as a regulator of the gastrointestinal tract's physiology, and further remodel the gut microbiota in favor of beneficial microbes. The inventors have surprisingly found that SCFAs, at gastrointestinal physiological levels, can inhibit the transfer of plasmids between bacteria.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The term “about”, as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the term “administering,” refers to the placement of a composition as disclosed herein into a subject by a method or route which results in at least partial delivery of the short chain fatty acid or short chain fatty acid-producing bacteria or at a desired site.

The term “bacterial conjugation” as used herein refers to the direct transfer of genetic material between at least two bacterial cells (also referred to as biological conjugation). Typically, bacterial conjugation requires cell-to-cell contact. The bacteria may be of the same species or of different species.

The term “bacterial horizontal gene transfer” as used herein refers to the direct transfer of genetic material between at least two bacterial cells, wherein the gene transfer is not via vertical transmission (i.e., is not the transmission of DNA from a parent to its offspring). Horizontal gene transfer is typically achieved by cell-to-cell contact (e.g., by bacterial conjugation). However, horizontal gene transfer may not involve cell-to-cell contact and may be achieved by transformation or by transduction.

As used herein, the terms “effective amount” refers to a dosage sufficient to inhibit bacterial conjugation or bacterial gene transfer. This can vary depending on the type of targeted bacteria, the subject to which the composition is administered, or the surface on which the bacteria reside. An effective amount can be determined by one of skill in the art especially in view of the disclosure provided below.

The phrase “pharmaceutically acceptable” as used herein refers to those compounds, materials, compositions, and/or dosage forms which are, within the scope of sound agricultural and/or animal husbandry industry standards and/or medical/veterinary judgment, suitable for use in contact with the tissues of the subject with toxicity, irritation, allergic response, or other problems or complications, commensurate with a reasonable benefit/risk ratio.

The term “subject” refers to any organism or animal subject that is an object of a method or material, including mammals, e.g., humans, laboratory animals (e.g., primates, rats, mice, rabbits), livestock (e.g., poultry, pigs, cows, sheep, and goats), household pets (e.g., dogs, cats, and rodents), and horses. Synonyms used herein include “patient” and “animal”.

The compositions of the present disclosure may comprise at least one short chain fatty acid. As used herein, the term “short chain fatty acid” or “SCFA” has its general meaning in the art and refers to aliphatic carboxylic acids composed of 1 to 6 carbon atoms, which may be linear or branched. Examples of short-chain fatty acids include: formic acid; acetic acid; propionic acid; butyric (butanoic) acid; isobutyric (2-methylbutanoic) acid; valeric (pentanoic) acid; isovaleric (3-methylbutanoic); and caproic (hexanoic) acid. In some embodiments, the SCFA is selected from saturated fatty acids comprising six or less carbon atoms, or five or less carbon atoms. In some embodiments, the SCFA is formic acid, acetic acid, propionic acid, butyric acid, isobutyric acid, valeric acid, isovaleric acid, or methylbutyric acid. In some embodiment, the SCFA is also a salt or an ester selected from formate, acetate, propionate, butyrate, isobutyrate, valerate, isovalerate, or methylbutyrate.

In certain embodiments, the composition of the disclosure has the SCFA or combination of SCFAs present in the amount of between about 0.01 wt-% and about 10 wt-%, between about 0.05 wt-% and about 5 wt-%, or between about 0.1 wt-% and about 2 wt-%. It is to be understood that all values and ranges between these values and ranges are encompassed by the present disclosure.

The compositions of the present disclosure may comprise a probiotic. As used herein the term “probiotic” is meant to designate live microorganisms which, they are integrated in a sufficient amount, exert a positive effect on health, comfort and wellness beyond traditional nutritional effects. In certain embodiments, the probiotic is a SCFA-producing bacteria. The probiotic bacteria can be from the same bacterial strain or a mixture of different bacterial strains, such as at least two, or at least three, or at least four, or at least five, or at least six, or at least seven, or at least eight, and so forth.

Non limiting examples of probiotics include: Bifidobacterium spp., Lactobacillus spp., Lactococcus spp., Enterococcus spp., in particular Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, or a combination thereof.

In certain embodiments, the probiotic bacteria are from the genus Bifidobacterium, including but not limited to, Bifidobacterium adolescentis, Bifidobacterium animalis, Bifidobacterium longum, Bifidobacterium breve, or Bifidobacterium bifidum.

In certain embodiments, the probiotic bacteria are from the genus Lactobacillus, including but not limited to, Lactobacillus acidophilus strain NP 28, Lactobacillus acidophilus strain NP51, Lactobacillus subsp. lactis strain NP7, Lactobacillus reuteri strain NCIMB 30242, Lactobacillus casei strain Shirota, Lactobacillus reuteri strain DSM 17938, Lactobacillus reuteri strain NCIMB 30242, Lactobacillus acidophilus strain NCFM, Lactobacillus rhamnosus strain HN001, Lactobacillus rhamnosus strain HN001, Lactobacillus reuteri strain DSM 17938, Lactobacillus casei, Lactobacillus casei subsp. rhamnosus strain GG, Lactobacillus acidophilus, Lactobacillus lactis, Lactobacillus acetotolerans, Lactobacillus acidifarinae, Lactobacillus acidipiscis, Lactobacillus alimentarius, Lactobacillus amylolyticus, Lactobacillus amylovorus, Lactobacillus aviaries, Lactobacillus brevis, Lactobacillus buchneri, Lactobacillus cacaonum, Lactobacillus casei subsp. casei, Lactobacillus collinoides, Lactobacillus composti, Lactobacillus coryniformis subsp. coryniformis, Lactobacillus crispatus, Lactobacillus crustorum, Lactobacillus curvatus, Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus dextrinicus, Lactobacillus diolivorans, Lactobacillus fabifermentans, Lactobacillus farciminis, Lactobacillus fermentum, Lactobacillus fructivorans, Lactobacillus frumenti, Lactobacillus gallinarum, Lactobacillus gasseri, Lactobacillus ghanensis, Lactobacillus hammesii, Lactobacillus harbinensis, Lactobacillus helveticus, Lactobacillus hilgardii, Lactobacillus homohiochii, Lactobacillus hordei, Lactobacillus jensenii, Lactobacillus johnsonii, Lactobacillus kefiri, Lactobacillus kefiranofadens subsp. kefiranofaciens, Lactobacillus kefiranofadens subsp. kefirgranum, Lactobacillus kimchii, Lactobacillus kisonensis, Lactobacillus mail, Lactobacillus manihotivorans, Lactobacillus mindensis, Lactobacillus mucosae, Lactobacillus nagelii, Lactobacillus namurensis, Lactobacillus nantensis, Lactobacillus nodensis, Lactobacillus oeni, Lactobacillus otakiensis, Lactobacillus panis, Lactobacillus parabrevis, Lactobacillus parabuchneri, Lactobacillus paracasei subsp. paracasei, Lactobacillus parakefiri, Lactobacillus paralimentarius, Lactobacillus paraplantarum, Lactobacillus pentosus, Lactobacillus perolens, Lactobacillus plantarum subsp. plantarum, Lactobacillus pobuzihii, Lactobacillus pontis, Lactobacillus rapi, Lactobacillus reuteri, Lactobacillus rhamnosus, Lactobacillus rossiae, Lactobacillus sakei subsp carnosus, Lactobacillus sakei subsp. sakei, Lactobacillus sali varius subsp. salivarius, Lactobacillus sanfranciscensis, Lactobacillus satsumensis, Lactobacillus secaliphilus, Lactobacillus senmaizukei, Lactobacillus siliginis, Lactobacillus spicheri, Lactobacillus suebicus, Lactobacillus sunkii, Lactobacillus tucceti, Lactobacillus vaccinostercus, Lactobacillus versmoldensis, or Lactobacillus yamanashiensis.

In certain embodiments, the probiotic bacteria are from the genus Lactococcus, including but not limited to Lactococcus lactis, Lactococcus lactis subsp. cremoris, Lactococcus lactis subsp. lactis, or Lactococcus raffinolactis.

In certain embodiments, the bacteria are from the genus Enterococcus, including but not limited to, Enterococcus durans, Enterococcus faecalis, or Enterococcus faecium.

In some embodiments, the composition comprises the probiotic in the amount of about 100 to about 10¹² colony forming unit (CFU), about 100 to about 10⁹ CFU, about 100 to about 10⁶ CFU, about 100 to about 10⁵ CFU, about 100 to about 10⁴ CFU, or about 100 to about 10³ CFU, or about 10³ to about 10¹² CFU, about 10³ to about 10⁹ CFU, about 10³ to about 10⁶ CFU, about 10³ to about 10⁵ CFU, or 10³ to about 10⁴ CFU, or 10⁴ to about 10¹² CFU, about 10⁴ to about 10⁹ CFU, about 10⁴ to about 10⁶ CFU, or 10⁴ to about 10⁵ CFU, or 10⁵ to about 10¹² CFU, about 10⁵ to about 10⁹ CFU, or 10⁵ to about 10⁶ CFU, or 10⁶ to about 10¹² CFU, about 10⁶ to about 10″ CFU, about 10⁶ to about 10¹⁰ CFU, about 10⁶ to about 10⁹ CFU, about 10⁶ to about 10⁸ CFU, or 10⁶ to about 10⁷ CFU, or 10⁷ to about 10¹² CFU, about 10⁷ to about 10″ CFU, about 10⁷ to about 10¹⁰ CFU, about 10⁷ to about 10⁹ CFU, or 10⁷ to about 10⁸ CFU, or 10⁸ to about 10¹² CFU, about 10⁸ to about 10¹¹ CFU, about 10⁸ to about 10¹⁰ CFU, or 10⁸ to about 10⁹ CFU, or 10⁹ to about 10¹² CFU, about 10⁹ to about 10¹¹ CFU, or about 10⁹ to about 10¹⁰ CFU.

Concerning the compositions of the disclosure, which may comprise different bacterial strains, any mixing ratio is possible. The mixing ratio is indicated in colony forming units (CFU), which are suitably determined prior to mixing the individual strains. The ratios of the strains may or may not be equal, such as 1:(0.1-10) for a composition comprising two strains, 1:(0.1-10):(0.1-10) for a composition comprising three strains, 1:(0.1-10):(0.1-10):(0.1-10) for a composition comprising four strains, and so forth. For example, the ratio in a composition comprising two strains may be from 1:2 to 2:1. In another embodiment, the ratios of the strains are roughly or substantially equal, such as 1:1 for a composition comprising two strains, 1:1:1 for a composition comprising three strains, 1:1:1:1 for a composition comprising four strains, and so forth. The composition can be prepared by mixing the respective bacterial amount (as determined by colony count) of each strain to be incorporated into the composition. The strains to be incorporated may be provided as stocks of individual strains, each one of them for example in the form of a lyophilizate. In the event that different stocks have different concentrations (CFU/g), appropriate amounts (g) of each one are used, so that the desired composition has the desired CFU of each of the strains.

The compositions of the present disclosure may comprise one or more prebiotics. Prebiotics are a category of functional food, defined as non-digestible food ingredients that beneficially affect the host by selectively stimulating the growth and/or activity of one or a limited number of bacteria in the intestines, e.g., ilea, duodenum, cecum, or colon, and thus improve host health. Prebiotics refer to food sources which can be metabolized by probiotics but are non-digestible or poorly digestible by an animal. Thus, following uptake by the animal, the non-digestible prebiotics can pass through the small intestine and enter the large intestine to stimulate the growth of the probiotics in this compartment. Prebiotics can thus serve as a food source for probiotics. Typically, prebiotics are carbohydrates (such as oligosaccharides). The most prevalent forms of prebiotics are nutritionally classed as soluble fiber. To some extent, many forms of dietary fiber exhibit some level of prebiotic effect.

Examples of suitable prebiotics include alginate, xanthan, pectin, locust bean gum (LBG), inulin, guar gum, galacto-oligosaccharide (GOS), fructo-oligosaccharide (FOS), polydextrose (i.e. Litesse®), lactitol, lactosucrose, soybean oligosaccharides, isomaltulose (Palatinose™), isomalto-oligosaccharides, gluco-oligosaccharides, xylo-oligosaccharides, mannan-oligosaccharides, arabino-xylo oligosaccharides, beta-glucans, cellobiose, raffinose, gentiobiose, melibiose, xylobiose, cyclodextrins, isomaltose, trehalose, stachyose, panose, pullulan, verbascose, galactomannans, and all forms of resistant starches. In certain embodiments, the prebiotic comprises fructo-oligosaccharides, galacto-oligosaccharides, mannan-oligosaccharides, or a combination thereof. Examples of dietary sources of prebiotics include soybeans, inulin sources (such as Jerusalem artichoke, jicama, and chicory root), raw oats, unrefined wheat, unrefined barley and yacon. Alternatively, prebiotics can be purified or chemically or enzymatically synthesized.

In certain embodiments, the composition of the disclosure has the prebiotic or combination of prebiotics present in the amount of between about 0.01 wt-% and about 25 wt-%, between about 0.05 wt-% and about 10 wt-%, or between about 0.1 wt-% and about 5 wt-%. It is to be understood that all values and ranges between these values and ranges are encompassed by the present disclosure.

While is it possible to administer the SCFA, the SCFA-producing probiotic bacteria, and/or the prebiotic alone (i.e. without any support, diluent or excipient), the compositions of the present disclosure may be administered on or in a support as part of a product, in particular as a component of a food product, an animal feed, a dietary supplement or a pharmaceutical formulation. These products typically contain additional components well known to those skilled in the art.

Any product which can benefit from the composition may be used. These include but are not limited to foods and pharmaceutical products. The term “food” is used in a broad sense—and covers food for humans as well as food for animals (i.e. a feed). In certain embodiments, the food is for human consumption. In certain embodiments, the composition may be used as, or in the preparation of, animal feeds, such as livestock feeds, in particular poultry (such as chicken) feed, or pet food. The food may be in the form of a solution or as a solid depending on the use and/or the mode of administration.

When used as, or in the preparation of, a food, the composition may be used in conjunction with one or more of: a nutritionally acceptable carrier, a nutritionally acceptable diluent, a nutritionally acceptable excipient, a nutritionally acceptable adjuvant, a nutritionally active ingredient.

The composition may be used as a food ingredient and/or feed ingredient. As used herein the term “food ingredient” or “feed ingredient” includes a formulation which is or can be added to functional foods or foodstuffs as a nutritional supplement. The composition may be, or may be added to, food supplements (also referred to as dietary supplements).

The composition may be, or may be added to, functional foods. As used herein, the term “functional food” means food which is capable of providing not only a nutritional effect but is also capable of delivering a further beneficial effect to consumer. Accordingly, functional foods are ordinary foods that have components or ingredients (such as those described herein) incorporated into them that impart to the food a specific functional (e.g., medical or physiological benefit) other than a purely nutritional effect. Some functional foods are nutraceuticals. As used herein, the term “nutraceutical” means a food which is capable of providing not only a nutritional effect and/or a taste satisfaction but is also capable of delivering a therapeutic (or other beneficial) effect to the consumer. Nutraceuticals cross the traditional dividing lines between foods and medicine.

In some embodiments, the composition may comprise one or more protein sources. As used herein, the term “protein” refers to both proteins derived from a source of protein, to peptides, and to free amino acids in general. There can be one or several protein sources. Protein sources based on whey, casein, and mixtures thereof may be used as well as protein sources based on soy.

In one embodiment, the composition comprises one or more vitamins. The vitamin can be fat-soluble or water soluble vitamins. Vitamins may be folic acid, vitamin B12 and vitamin B6, in particular folic acid and vitamin B12, in particular folic acid. In some embodiments, the composition comprises one or more vitamin which is lipid-soluble, for example one or more of vitamin A, vitamin D, vitamin E, and vitamin K. Suitable forms of any of the foregoing are salts of the vitamin, derivatives of the vitamin, compounds having the same or similar activity of the vitamin, and metabolites of the vitamin.

In some embodiments, the composition further comprises one or more minerals. Examples of minerals are sodium, potassium, chloride, calcium, phosphate, magnesium, iron, zinc, copper, selenium, manganese, fluoride, iodine, chromium, or molybdenum. The minerals are usually added in salt form. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof. The minerals may be added alone or in combination.

In some embodiments, the composition comprises at least one lipid. As used herein, a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In other embodiments, the composition comprises at least one modified lipid, for example, a lipid that has been modified by cooking.

In some embodiments, the composition comprises an emulsifier. Examples of food grade emulsifiers typically include diacetyl tartaric acid esters of mono- and di-glycerides, lecithin and mono- and di-glycerides. Similarly suitable salts and stabilizers may be included.

In some embodiments, the composition contains protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional additives and adjuvants, excipients and diluents, including, but not limited to, water, gelatine of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. In all cases, such further components will be selected having regard to their suitability for the intended recipient.

The compositions disclosed herein may include one or more excipients. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the composition comprises a disintegrant as an excipient. In other embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In another embodiment, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In other embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

The weight percent of the excipient or combination of excipients in the composition may be about 99% or less, such as about 95% or less, about 90% or less, about 85% or less, about 80% or less, about 75% or less, about 70% or less, about 65% or less, about 60% or less, about 55% or less, 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

In certain embodiments, the compositions of the present disclosure are mixed with animal feed. In some embodiments, animal feed may be present in various forms such as pellets, capsules, granulated, powdered, mash, liquid, or semi-liquid.

In some embodiments, compositions of the present disclosure are mixed into the premix or mash at the feed mill, alone as a standalone premix, and/or alongside other feed additives. In one embodiment, the compositions of the present disclosure are mixed into or onto the feed at the feed mill. In another embodiment, compositions of the present disclosure are mixed into the feed itself.

In some embodiments, feed of the present disclosure may be supplemented with water, premix or premixes, forage, fodder, beans (e.g., whole, cracked, or ground), grains (e.g., whole, cracked, or ground), bean- or grain-based oils, bean- or grain-based meals, bean- or grain-based haylage or silage, bean- or grain-based syrups, fatty acids, sugar alcohols (e.g., polyhydric alcohols), commercially available formula feeds, oyster shells and those of other bivalves, and mixtures thereof.

In some embodiments, forage encompasses hay, haylage, and silage. In some embodiments, hays include grass hays (e.g., sudangrass, orchardgrass, or the like), alfalfa hay, and clover hay. In some embodiments, haylages include grass haylages, sorghum haylage, and alfalfa haylage. In some embodiments, silages include maize, oat, wheat, alfalfa, clover, and the like.

In some embodiments, premix or premixes may be utilized in the feed. Premixes may comprise micro-ingredients such as vitamins, minerals, amino acids; chemical preservatives; pharmaceutical compositions such as antibiotics and other medicaments; fermentation products, and other ingredients. In some embodiments, premixes are blended into the feed.

In some embodiments, the feed may include feed concentrates such as soybean hulls, soybean oils, sugar beet pulp, molasses, high protein soybean meal, ground corn, shelled corn, wheat midds, distiller grain, cottonseed hulls, and grease.

In some embodiments, feed occurs as a compound, which includes, in a mixed composition capable of meeting the basic dietary needs, the feed itself, vitamins, minerals, amino acids, and other necessary components. Compound feed may further comprise premixes.

In some embodiments, compositions of the present disclosure may be mixed with animal feed, premix, and/or compound feed. Individual components of the animal feed may be mixed with the probiotic compositions prior to feeding to poultry. The composition may be applied into or on a premix, into or on a feed, and/or into or on a compound feed.

The compositions may be incorporated into feed prior to, during, or after feed processing. For example, the compositions may be dusted on the feed product after the pelleting stage. Importantly, at least a portion of the bacteria added to the feed must be able to survive any aspect of feed processing and preparation that they are subjected to (e.g., exposure to high temperatures used in the pelleting stage).

In some embodiments, the composition of the disclosure comprises a direct-fed microbial (DFM) composition. As used herein, a “direct-fed microbial” refers to live (viable) microorganisms that are supplied through feed. A DFM composition may be added to an animal's diet continuously and/or administered as a bolus at particular developmental stage (e.g., at birth, at weaning).

The compositions of the present disclosure may be used as, or in the preparation of, a pharmaceutical. Here, the term “pharmaceutical” is used in a broad sense—and covers pharmaceuticals for humans as well as pharmaceuticals for animals (i.e. veterinary applications). In certain embodiments, the pharmaceutical is for human use and/or for animal husbandry.

A pharmaceutically acceptable support may be, for example, a support in the form of compressed tablets, tablets, capsules, ointments, suppositories or drinkable solutions. Other suitable forms are provided below.

When used as, or in the preparation of, a pharmaceutical, the composition may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient. The pharmaceutical may be in the form of a solution or as a solid depending on the use and/or the mode of administration.

The compositions of the disclosure may include different types of carriers depending on whether it is to be administered in solid, liquid or aerosol form as would be known to one of ordinary skill in the art (see, for example, Remington's Pharmaceutical Sciences, 18th Ed. Mack Printing Company, 1990, incorporated herein by reference).

The composition may be used in the form of solid or liquid preparations or alternatives thereof. Examples of solid preparations include, but are not limited to tablets, capsules, dusts, granules, and powders which may be wettable, spray-dried or freeze-dried. Examples of liquid preparations include, but are not limited to, aqueous, organic or aqueous-organic solutions, suspensions, and emulsions.

Suitable examples of forms include one or more of: tablets, pills, capsules, ovules, solutions or suspensions, which may contain flavoring or coloring agents, for immediate-, delayed-, modified-, sustained-, pulsed- or controlled-release applications.

A capsule typically comprises a core material comprising the composition of the disclosure and a shell wall that encapsulates the core material. In some embodiments, the core material comprises at least one of a solid, a liquid, and an emulsion. In other embodiments, the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In yet other embodiments, at least one polymer functions as taste-masking agents.

Tablets, pills, and the like can be compressed, multiply compressed, multiply layered, and/or coated. The coating can be single or multiple. In one embodiment, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In another embodiment, the coating material comprises at least one of a fat and an oil. In other embodiments, the at least one of a fat and an oil is high temperature melting. In yet another embodiment, the at least one of a fat and an oil is hydrogenated or partially hydrogenated. In one embodiment, the at least one of a fat and an oil is derived from a plant. In other embodiments, the at least one of a fat and an oil comprises at least one of glycerides, free fatty acids, and fatty acid esters. In some embodiments, the coating material comprises at least one edible wax. The edible wax can be derived from animals, insects, or plants. Non-limiting examples include beeswax, lanolin, bayberry wax, carnauba wax, and rice bran wax. Tablets and pills can additionally be prepared with enteric coatings.

Alternatively, powders or granules embodying the compositions disclosed herein can be incorporated into, for example, a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product can be a solid foodstuff. Suitable examples of a solid foodstuff include without limitation a food bar, a snack bar, a cookie, a brownie, a muffin, a cracker, an ice cream bar, a frozen yogurt bar, and the like.

In other embodiments, the compositions of the disclosure are incorporated into a therapeutic food. In some embodiments, the therapeutic food is a ready-to-use food that optionally contains some or all essential macronutrients and micronutrients. In another embodiment, the compositions of the disclosure are incorporated into a supplementary food that is designed to be blended into an existing meal. In one embodiment, the supplemental food contains some or all essential macronutrients and micronutrients. In another embodiment, the compositions of the disclosure are blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

For aqueous suspensions, the composition may be combined with various sweetening or flavoring agents, coloring matter or dyes, with emulsifying and/or suspending agents and with diluents such as water, propylene glycol and glycerin, and combinations thereof.

Agriculturally acceptable carriers include, for example, mineral earth (e.g, silicas, lime, calcium carbonate, chalk, clay, etc.) and crushed products of vegetable origin (e.g, cereal meal, wood meal, rice hulls, wheat bran, cellulose powders, etc.).

For oral administration, in some embodiments, the compositions of the disclosure may be in a lyophilized or dried form, e.g., a dried powder. In some embodiments, the compositions are formulated as a dry powder that is soluble in water or an organic solvent, or may be directly added to an animal feed during processing or manufacturing. In other embodiments, the compositions may be in a liquid form, e.g, a suspension or solution. In some embodiments, the compositions are formulated for example, in the form of a paste, cream, a gel, a capsule, an aerosol spray, or a tablet. In some embodiments, the compositions are formulated to be delivered by oral gavage or drenched. In some embodiments, the compositions are encased in a coating formulated for release in the gastrointestinal tract. In some embodiments, the compositions are provided in water consumed by the animal.

The present disclosure provides the use of the compositions of the disclosure in a method of inhibiting bacterial conjugation or horizontal gene transfer in the gut of a subject.

As used herein, the term “inhibiting” refers to preventing, arresting, or reducing the occurrence of bacterial conjugation or bacterial horizontal gene transfer.

In certain embodiments, inhibiting bacterial conjugation is by at least about 10%, 20%, 30%, 40%, 50%, 60%, 70%, 80%, 90% or by 100% as compared to bacteria not treated by the composition comprising a SCFA and/or a SCFA-producing probiotic bacterium. Those of skill in the art will understand that various methodologies and assays can be used to assess inhibition of bacterial conjugation. In certain embodiments, assessing inhibition of bacterial conjugation is carried out by assessing the conjugation frequency, e.g., by calculating the ratio between the obtained transconjugant CFU and the number of bacterial donors that were used in the conjugation assay.

In certain embodiments, inhibiting bacterial horizontal gene transfer is by at least about 10%, 20%, 30%, 40%, 50%, 60%, 70%, 80%, 90% or by 100% as compared to bacteria not treated by the composition comprising a SCFA and/or a SCFA-producing probiotic bacterium. Those of skill in the art will understand that various methodologies and assays can be used to assess inhibition of bacterial horizontal gene transfer. In certain embodiments, assessing inhibition of bacterial horizontal gene transfer is carried out by, e.g., PCR, southern blot, sequence composition methods or by homology methods.

In certain embodiments, the levels of SCFAs in the gut of a subject are increased by 10-fold to 10,000-fold following administration of the composition. In some embodiments, the levels of SCFAs are increased by 10-fold to 1,000-fold following administration of the composition. In some embodiments, the levels of SCFAs are increased by 2-fold to 100-fold following administration of the composition. In some embodiments, the levels of SCFAs are increased by 2-fold, 5-fold, 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 1,000-fold following administration of the composition.

The main mechanism by which bacteria acquire resistance is by the acquisition of antimicrobial resistance (AMR) genes by bacterial conjugation and/or horizontal gene transfer (e.g., via plasmid transfer).

In certain embodiments, inhibiting bacterial conjugation or horizontal gene transfer blocks genetic transfer between bacteria including transfer of, for example, antibiotic resistant genes (e.g., tetA, ampC), adhesion factors (e.g., fimbria clusters), invasion factors (e.g., Type 3 secretion system), toxins (e.g., stx), metabolic genes (e.g., mal genes used for maltose metabolism), virulence factors (e.g., ybt), and tolerance to environmental stresses genes (e.g., mer genes). In certain embodiments, inhibiting bacterial conjugation or horizontal gene transfer blocks genetic transfer between bacteria including acquisition of antibiotic resistance genes (e.g., those coding for multidrug resistance (MDR)). In certain embodiments, inhibiting bacterial conjugation blocks genetic transfer between bacteria including the transfer of plasmids.

By inhibiting bacterial conjugation and/or horizontal gene transfer, the compositions of the disclosure can be used to reduce the spread of antibiotic resistance genes and virulence factors between bacteria by e.g., conjugation, and accordingly increase susceptibility of pathogenic bacteria to treatment.

Thus, according to another embodiment there is provided a method of increasing susceptibility of pathogenic bacteria to antibiotic treatment, the method comprising contacting the pathogenic bacteria with an effective amount of a composition of the disclosure, thereby increasing susceptibility of the pathogenic bacteria to the antibiotic treatment.

In certain embodiments, the pathogenic bacteria are enteropathogenic bacteria. Exemplary enteropathogenic bacteria include, but are not limited to, Salmonella enterica (e.g., S. typhi and S. typhimurium), Shigella (S. flexneri and S. dysenteriae), Vibrio cholerae/parahaemolyticus, Escherichia (e.g., E. coli, E. coli 0157:H7), Campylobacter jejuni, Listeria monocytogenes, Klebsiella spp., Proteos spp., Clostridium difficile, Bacillus cereus and Helicobacter pylori. In certain embodiments, the bacteria are resistant to multiple antimicrobial treatments (i.e. multidrug resistant (MDR)).

The compositions of the disclosure can be administered enterally, in other words, by a route of access to the gastrointestinal tract. This includes oral administration, rectal administration (including enema, suppository, or colonoscopy), or by an oral or nasal tube (nasogastric, nasojejunal, oral gastric, or oral jejunal).

The composition can be administered to at least one region of the gastrointestinal tract, including the mouth, esophagus, stomach, small intestine, large intestine, and rectum. In some embodiments, it is administered to all regions of the gastrointestinal tract. The compositions can be administered orally in the form of medicaments such as powders, capsules, tablets, gels or liquids. The compositions can also be administered in gel or liquid form by the oral route or through a nasogastric tube, or by the rectal route in a gel or liquid form, by enema or instillation through a colonoscope or by a suppository.

If the composition is administered colonoscopically and, optionally, if the composition is administered by other rectal routes (such as an enema or suppository) or even if the subject has an oral administration, the subject can have a colon-cleansing preparation. The colon-cleansing preparation can facilitate proper use of the colonoscope or other administration devices, but even when it does not serve a mechanical purpose, it can also maximize the proportion of the composition relative to the other organisms previously residing in the gastrointestinal tract of the subject. For example, the colon cleansing preparation may maximize the amount of bacteria of the composition that reach and/or engraft in the gastrointestinal tract of the subject. Any ordinarily acceptable colon-cleansing preparation may be used such as those typically provided when a subject undergoes a colonoscopy.

In some embodiments, the subject has not received antibiotics in advance of treatment with the compositions. In other embodiments, the subject has not previously received an antibiotic compound in the one month prior to administration of the composition. In other embodiments, the subject has received one or more treatments with one or more different antibiotic compounds.

In some embodiments, the composition is administered only once. In some embodiments, the composition is administered at intervals greater than two days, such as once every three, four, five or six days, or every week or less frequently than every week. In other embodiments, the preparation can be administered intermittently according to a set schedule, e.g., once a day, once weekly, or once monthly. In another embodiment, the preparation may be administered on a long-term basis to subjects who are at risk for infection with or who may be carriers of pathogens, including subjects who will have an invasive medical procedure (such as surgery), who will be hospitalized, who live in a long-term care or rehabilitation facility, who are exposed to pathogens by virtue of their profession (livestock and animal processing workers), or who could be carriers of pathogens (including hospital workers such as physicians, nurses, and other health care professionals).

In certain embodiments, the methods of the disclosure may be carried out on a subject with a particular profile. For example, 16S rRNA sequencing may be performed for a given subject to identify the bacteria present in their microbiota. The sequencing may either profile the entire microbiome using 16S rRNAsequencing (to the family, genera, or species level), a portion of the subject's microbiome using 16S rRNA sequencing, or it may be used to detect the presence or absence of specific candidate bacteria that are biomarkers for health or a particular disease state, such as markers of multi-drug resistant organisms or specific genera of concern, such as Escherichia coli.

The compositions of the present disclosure may be administered to any animal, including, for example, livestock (including cattle, horses, pigs, poultry, and sheep), and humans. As used herein the term “poultry” relates to the class of domesticated fowl (birds) used for food or for their eggs. Poultry includes wildfowl, waterfowl, and game birds. Examples of poultry include, but are not limited to, chicken, broilers, bantams, turkey, duck, geese, guinea fowl, peafowl, quail, dove, pigeon (squab), and pheasant. In certain embodiments, the animal is a companion animal (including pets), such as a dog or a cat for instance. In certain embodiments, the subject may suitably be a human.

Any route of administration is suitable, but oral administration may be preferred. Most typically, a dose is given to every animal directly into the mouth to make sure that the animal swallows the dose. Alternatively, the composition may also be provided as a food supplement, i.e. added to the daily feed of the animal.

To provide for easy use, the composition may be in dosed form. A dose may have a volume in the range of about 0.1 ml to about 100 ml, about 0.2 ml to about 50 ml, about 0.5 ml to about 20 ml, about 1 ml to about 10 ml, about 2 ml to about 8 ml, or about 5 ml.

Any number of doses may be administered, and the skilled person can choose the length of the treatment according to the needs at the respective farm. In certain embodiments, the total number of doses administered to an animal is 10 or less, such as any number selected from the following: 1, 2, 3, 4, 5, 6, 7, 8, 9 10, or any range combining any one of these numbers (except 10) with any one of these number, provided that the second number is higher (e.g., 1 to 3 doses).

The compositions of the disclosure can be useful in a variety of clinical situations. For example, the compositions can be administered as a complementary treatment to antibiotics when a patient is suffering from an acute infection, to reduce the risk of acquisition of antimicrobial resistance genes, or when a patient will be in close proximity to others with or at risk of infections (physicians, nurses, hospital workers, family members of those who are ill or hospitalized).

The compositions of the disclosure can be administered with other agents in a combination therapy mode, including anti-microbial agents. Administration can be sequential, over a period of hours or days, or simultaneous.

In one embodiment, the compositions of the disclosure are included in combination therapy with one or more anti-microbial agents, which include anti-bacterial agents, anti-fungal agents, anti-viral agents, and anti-parasitic agents.

The present disclosure provides the use of the compositions of the disclosure in a method of inhibiting bacterial conjugation or horizontal gene transfer on a surface, the method comprising contacting the surface with a SCFA composition of the disclosure.

As used herein, the term “contacting” refers to the positioning of the SCFA (or compositions comprising same) of the disclosure such that they are in direct or indirect contact with the bacterial cells in such a way that the SCFA is able to inhibit or prevent bacterial conjugation. Thus, the present disclosure contemplates applying the SCFA to a desirable surface (e.g., one which bacterial cells may grow) and/or directly to the bacterial cells (e.g., to a surface on which bacterial cells have been identified).

The surface can be contacted with the SCFA (or compositions comprising same) using any method known in the art including spraying, spreading, wetting, immersing, dipping, painting, or adhering.

Any surface in which bacteria propagate can be contacted with the SCFA compositions of the disclosure. Bacteria can live and proliferate as individual cells in the environment (e.g., on surfaces) or they can grow as highly organized, multicellular communities encased in a self-produced polymeric matrix in close association with surfaces and interfaces, named biofilms.

In certain embodiments, the surface does not comprise bacteria (and the treatment is a preventive measure). In certain other embodiments, the surface already has bacteria present.

In certain embodiments, the surface is a medical surface or the surface of a medical device. As used herein the term “medical device” refers to any implant, instrument, apparatus, implement, machine, device or any other similar or related object (including any component or accessory), which is intended for use in the diagnosis, treatment, cure or prevention of disease or other conditions. Such medical device is intended for use in man or other animals and is anticipated to affect the structure or any function of the body. Such a medical device does not achieve its primary intended purposes through chemical action and is not dependent upon being metabolized for the achievement of its primary intended purposes.

Medical surfaces that may be applied (e.g., coated) with the SCFA compositions of the disclosure include, but not are limited to, artificial blood vessels, catheters and other devices for the removal or delivery of fluids to patients, artificial hearts, artificial kidneys, orthopedic pins, prosthetic joints, plates and implants; catheters and other tubes (including urological and biliary tubes, endotracheal tubes, peripherally insertable central venous catheters, dialysis catheters, long term tunneled central venous catheters, peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, aneurysm repair devices, mechanical heart valves, artificial joints, artificial larynxes, otological implants), anastomotic devices, vascular catheter ports, vascular stents, clamps, surgical staples, embolic devices, wound drain tubes, ocular lenses, dental implants, hydrocephalus shunts, pacemakers and implantable defibrillators, needleless connectors, voice prostheses, and the like.

Another application of the SCFA compositions of the disclosure is to apply the same to surfaces found in the medical and dental environments. Such surfaces include the inner and outer aspects of various instruments and devices, whether disposable or intended for repeated uses. Such surfaces include the entire spectrum of articles adapted for medical use, including without limitation, endoscopes, scalpels, needles, scissors and other devices used in invasive surgical, therapeutic or diagnostic procedures; blood filters. Other examples will be readily apparent to practitioners in these arts.

Surfaces found in the medical environment also include the inner and outer aspects of pieces of medical equipment, medical gear worn or carried by personnel in the health care setting. Such surfaces can include surfaces intended as biological barriers to infectious organisms in medical settings, such as gloves, aprons and face shields. Commonly used materials for biological barriers are thermoplastic or polymeric materials such as polyethylene, dacron, nylon, polyesters, polytetrafluoroethylene, polyurethane, latex, silicone and vinyl. Other surfaces can include counter tops and fixtures in areas used for medical procedures or for preparing medical apparatus, tubes and canisters used in respiratory treatments, including the administration of oxygen, of solubilized drugs in nebulizers and of anesthetic agents. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered.

Medical surfaces according to the present disclosure can also include laboratory articles including, but not limited to, microscopic slide, a culturing hood, a Petri dish or any other suitable type of tissue culture vessel or container known in the art.

In certain embodiments, the surface is a surface of farm animal housing. As used herein, the term “farm animal housing” refers to any area in which the farm animal is breed, raised, transported or slaughtered. For example, a farm animal housing may include an animal feeding operation, open barns, indoor barns, crates, cages, stalls, coops and trucks (e.g., used for transportation of farm animals). Furthermore, the term “farm animal housing” includes milking areas (e.g., of cows, sheep and goats) and egg laying areas.

In certain embodiments, the surface includes at least one of a cage, a crate, a floor, a wall, a ceiling, a door, a shelf, a fabric, a milking device, a collection tank (e.g., milking tank), a feeding device or utensil (e.g., a feeding trough, a feeding cup, a watering cup or watering trough), or a laying surface.

In certain embodiments, the composition comprises a SCFA and a disinfectant. In certain embodiments, the disinfectant comprises an alcohol, a chlorine, a chlorine compound, an aldehyde, an oxidizing agent, an iodine, an iodophor, an ozone, a phenolic, a quaternary ammonium compound, or a mixture of two or more thereof. In certain embodiments, the disinfectant comprises phenolic compounds (e.g., Pine-sol, One Stroke, Osyl), iodine or iodophors, (e.g., Betadine and Weladol), chlorine compounds (e.g., Clorox, generic bleach), quaternary ammonium compound (e.g., Roccal D Plus), alcohol-based compounds (e.g., Terralin), oxidizing compounds (e.g., Virkon S, Oxy-Sept 333), aldehyde compounds (e.g., Cidex®, Endosporine), peracetic acid (PAA) compounds (e.g., Nu Cidex®, Anioxyde 1000®, Hydraseptic®, Peralkan®). In certain embodiments, the disinfectant may comprise formaldehyde, ortho-phthalaldehyde, glutaraldehyde, silver dihydrogen citrate, polyaminopropyl biguanide, sodium bicarbonate, lactic acid, chlorine bleach, or a mixture of two or more thereof. In certain embodiments, the disinfectant may comprise methanol, ethanol, n-propanol, 1-propanol, 2-propanol, isopropanol, or a mixture of two or more thereof. In certain embodiments, the disinfectant may comprise a hypochlorite, chlorine dioxide, a dichloroisocyanurate, a monochloroisocyanurate, a halogenated hydantoin, or a mixture of two or more thereof. In certain embodiments, the disinfectant may comprise sodium hypochlorite, calcium hypochlorite, sodium dichloroisocyanurate, sodium chlorite, N-chloro-4-methylbenzenesulfonamide sodium salt, 2,4-dichorobenzyl alcohol, or a mixture of two or more thereof. In certain embodiments, the disinfectant may comprise performic acid, potassium permanganate, potassium peroxymonosulfate, or a mixture of two or more thereof. In certain embodiments, the disinfectant may comprise phenol, o-phenylphenol, chloroxylenol, hexachlorophene, thymol, amylmetacresol, or a mixture of two or more thereof. In certain embodiments, the disinfectant may comprise benzalkonuim chloride, cetyltrimethyl ammonium bromide, cetylpyridinium chloride, benzethonium chloride, boric acid, Brilliant green, chlorhexidine gluconate, tincture of iodine, providone-iodine, mercurochrome, manuka honey, octenidine dihydrochloride, polyhexamethylene biguamide, balsam of Peru, or a mixture of two or more thereof. In certain embodiments, the disinfectant may comprise peroxide, such as hydrogen peroxide, organic peroxides, peroxy acids, organic hydroperoxides, inorganic peroxides such as peroxide salts, acid peroxides, and mixtures of two or more thereof.

The compositions of the disclosure may further comprise one or more ingredients selected from water, a stabilizing agent, a surfactant (e.g., to provide the aqueous composition with surface active properties), a pH adjuster, one or more corrosion inhibitors (e.g., to provide corrosion inhibiting properties), and/or one or more chelators (e.g., to provide chelation capacity e.g., water softening).

In certain embodiments, the surfactant may comprise any compound that lowers surface tension or provides greater wettability. The surfactant may comprise one or more detergent, wetting agents, emulsifiers, foaming agents and/or dispersants. The surfactant may comprise one or more organic compounds that contain both hydrophobic groups and hydrophilic groups. The surfactant may comprise both a water insoluble component and a water soluble component. The surfactant may comprise one or more anionic, cationic, zwitterionic and/or nonionic compounds. The surfactant may comprise one or more alkanolamines, alkylarylsulfonates, amine oxides, poly(oxyalkylene)s, block copolymers comprising alkylene oxide repeat units, carboxylated alcohol ethoxylates, ethoxylated alcohols, alkyl phenols, ethoxylated alkyl phenols, ethoxylated amines, ethoxylated amides, oxiranes, ethoxylated fatty acids, ethoxylated fatty esters, ethoxylated oils, fatty esters, fatty acid amides, glycerol esters, glycol esters, sorbitan, sorbitan esters, imidazolines, lecithin, lignin, glycerides (e.g., mono-, di- and/or triglyceride), olefin sulfonates, phosphate esters, ethoxylated and/or propoxylated fatty acids and/or alcohols, sucrose esters, sulfates and/or alcohols and/or ethoxylated alcohols of fatty esters, sulfonates of dodecyl and/or tridecyl benzenes, sulfosuccinates, dodecyl and/or tridecyl benzene sulfonic acids, mixtures of two or more thereof, and the like. The surfactant may comprise ethanolamine, triethanolamine, octyldimethylamine oxide, nonylphenoxy poly(ethyleneoxy)ethanol, polyalkylene glycol, or a mixture of two or more thereof.

Also provided is a method of assaying a decrease in conjugation frequency of bacteria, the method comprising: (a) contacting a bacteria with a composition of the disclosure; (b) incubating the bacteria of step (a) with other bacteria; and (c) measuring conjugation frequency between the bacteria of step (a); and the other bacteria, wherein a decrease in conjugation frequency is determined when a lower conjugation frequency is measured as compared to a conjugation frequency in the absence of the SCFA.

In certain embodiments, conjugation frequency is calculated as the ratio between the obtained transconjugant colony forming units (CFU) and the number of bacterial donors that were used in the conjugation assay. A transconjugant refers to bacteria that have accepted a genetic material (e.g., plasmid DNA) from other donor bacteria via bacterial conjugation.

In certain embodiments, a decrease in conjugation frequency is by at least about 10%, 20%, 30%, 40%, 50%, 60%, 70%, 80%, 90% or by 100% as compared to bacteria not treated by the composition of the disclosure.

The following numbered embodiments also form part of the present disclosure:

1. A method of inhibiting bacterial conjugation or horizontal gene transfer in the gut of a subject, the method comprising: administering to the subject a composition comprising a prebiotic, wherein at least one short chain fatty acid is produced in the gut of the subject.

2. The method of embodiment 1, wherein the composition further comprises a short chain fatty acid-producing probiotic bacterium.

3. The method of embodiment 2, wherein the probiotic bacterium comprises a Lactobacillus spp.

4. The method of any one of embodiments 1-3, wherein the prebiotic comprises fructo-oligosaccharides, galacto-oligosaccharides, mannan-oligosaccharides, or a combination thereof.

5. The method of any one embodiments 1-4, wherein the short chain fatty acid comprises formic acid, acetic acid, propionic acid, butyric acid, isobutyric acid, valeric acid, isovaleric acid, methylbutyric acid, or a combination thereof.

6. The method of any one of embodiments 1-5, wherein the composition further comprises a carrier.

7. The method of any one of embodiments 1-6, wherein the composition further comprises an excipient.

8. The method of any one of embodiments 1-7, wherein the composition is administered orally.

9. The method of any one of embodiments 1-8, wherein the composition is administered with food or feed.

10. The method of any one of embodiments 1-9, wherein the subject is a poultry animal, a pig, a cow, or a human.

11. A method of inhibiting bacterial conjugation or horizontal gene transfer in the gut of a subject, the method comprising: administering to the subject a composition comprising at least one short chain fatty acid.

12. The method of embodiment 11, wherein the short chain fatty acid comprises formic acid, acetic acid, propionic acid, butyric acid, isobutyric acid, valeric acid, isovaleric acid, methylbutyric acid, or a combination thereof.

13. The method of embodiment 11 or embodiment 12, wherein the composition further comprises a carrier.

14. The method of any one of embodiments 11-13, wherein the composition further comprises an excipient.

15. The method of any one of embodiments 11-14, wherein the composition is administered orally.

16. The method of any one of embodiments 11-15, wherein the composition is administered with food or feed.

17. The method of any one of embodiments 11-16, wherein the subject is a poultry animal, a pig, a cow, or a human.

18. A composition for inhibiting bacterial conjugation or horizontal gene transfer comprising: at least one short chain fatty acid and/or a short chain fatty acid-producing probiotic bacterium.

19. The composition of embodiment 19, further comprising a prebiotic, wherein the prebiotic comprises fructo-oligosaccharides, galacto-oligosaccharides, mannan-oligosaccharides, or a combination thereof.

20. The composition of embodiment 19 or embodiment 10, wherein the probiotic bacterium comprises a Lactobacillus spp.

21. The composition of any one of embodiments 19-20, wherein the composition further comprises a carrier.

22. The composition of any one of embodiments 19-21, wherein the composition further comprises an excipient.

23. The composition of any one of embodiments 19-22, wherein the composition is formulated for oral administration.

24. The composition of any one of embodiments 19-23, wherein the composition is a food or feed composition.

25. A method of inhibiting bacterial conjugation or horizontal gene transfer on a surface, the method comprising: contacting the surface with a composition comprising at least one short chain fatty acid.

26. The method of claim 25, wherein the short chain fatty acid comprises formic acid, acetic acid, propionic acid, butyric acid, isobutyric acid, valeric acid, isovaleric acid, methylbutyric acid, or a combination thereof.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Inhibition of Conjugation by SCFAs

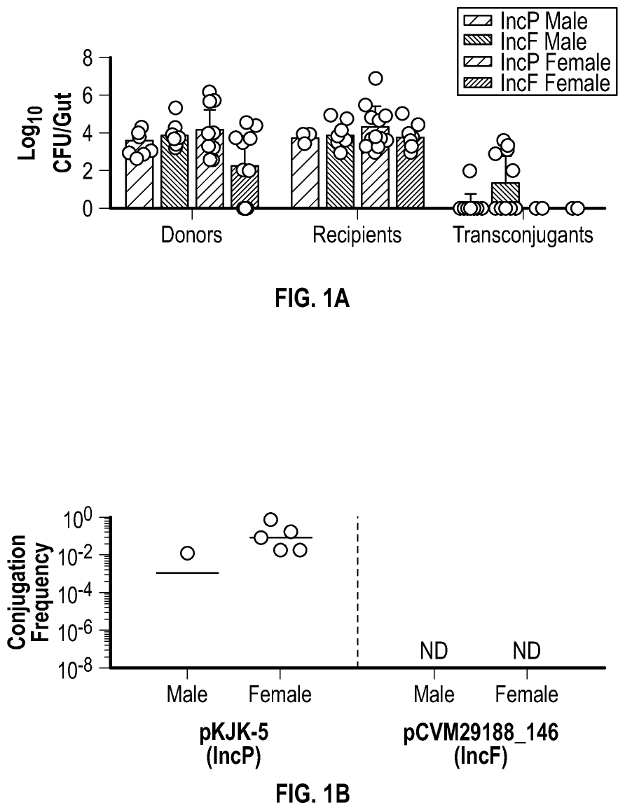

In vivo transfer of a conjugative large antibiotic resistance plasmids (LARPs) of varying incompatibility types; IncP (broad host range), IncF, IncI, and IncK (narrow host range, Enterobacteriaceae) between bacteria were conducted. Adult W¹¹¹⁸ flies (n=12) were separately fed recipient and donor strains. Flies were surface sterilized, homogenized, serially diluted, bacterially enumerated. Donors, recipients, and transconjugants were enumerated for total colony forming units (FIG. 1A) and conjugation frequency (FIG. 1B). Transconjugants were detected in one male and five female guts of the IncP plasmid type, however, no transconjugants were detected in the IncF (FIG. 1), IncI or the IncK plasmid types. Conjugations on the surface of fly media in the absence of flies resulted in the same detection of transconjugants in the IncP groups and none in IncF, IncI, or IncK groups.

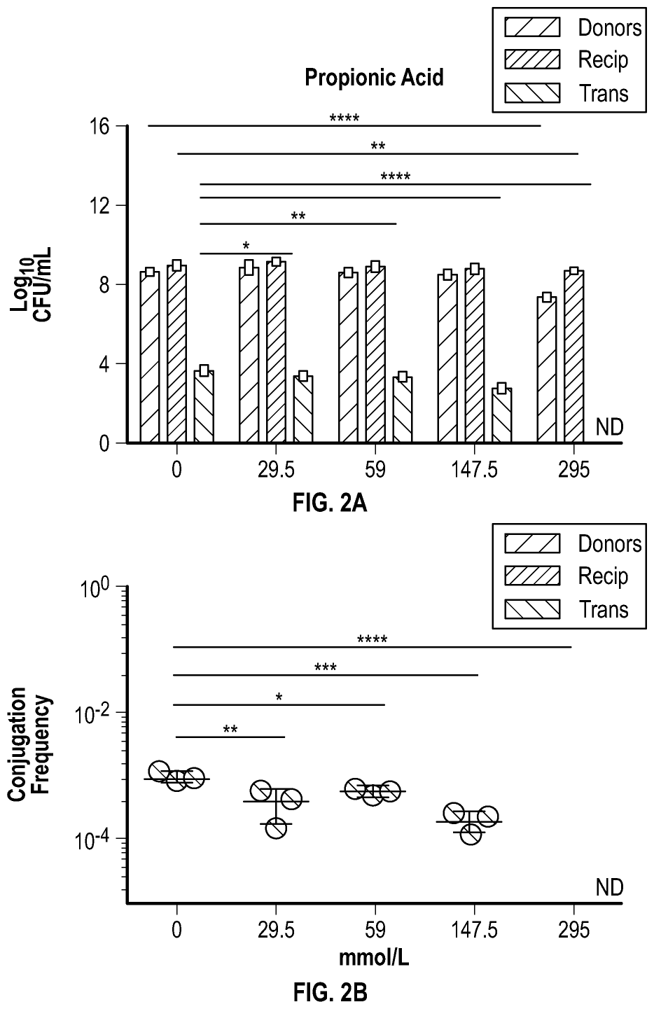

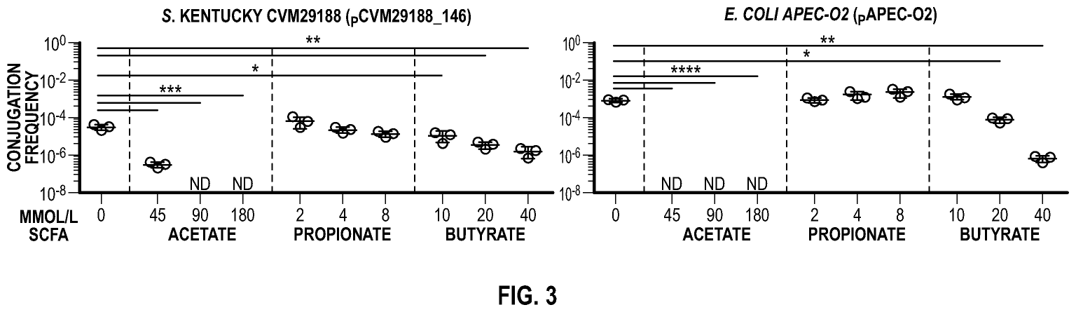

Propionic acid used in Drosophila media (FIG. 2) and physiological concentrations of SCFAs in chicken ceca (FIG. 3) were tested for inhibitory effects on conjugation in vitro. Propionate was added to in vitro broth conjugations between E. coli MG1655 (pCVM29188_146) and E. coli HS-4 at fly media concentrations. Propionic acid at as low as half the concentrations of standard Drosophila fly media, resulted in significant decreases in conjugation frequency (transconjugants divided by donors) (FIG. 2). Physiological concentrations of SCFAs in the chicken gut were added to broth conjugations with the conjugation pairs 1) S. Kentucky CVM29188 to E. coli HS-4, and 2) E. coli APEC-02 to E. coli HS-4 (FIG. 3). Acetate and butyrate significantly reduced transconjugant populations but not donor and recipient strains, however physiological concentrations of propionate did not significantly reduce conjugation.

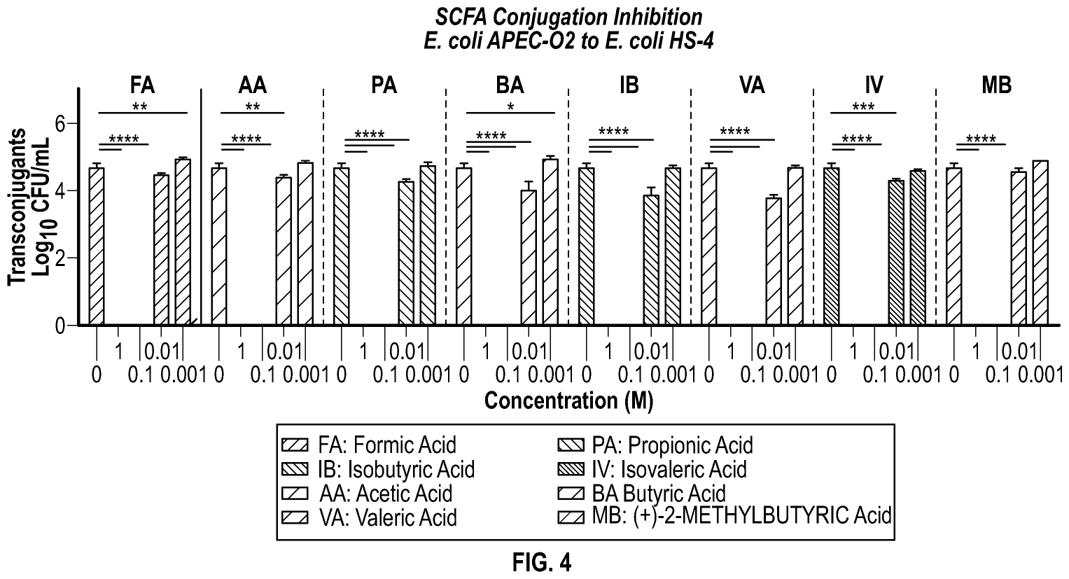

Eight distinct SCFAs (formic acid, acetic acid, propionic acid, butyric acid, isobutyric acid, valeric acid, isovaleric acid, and methylbutyric acid) were tested for inhibitory effects on conjugation in vitro. The SCFAs were added to broth conjugations with the conjugation pair E. coli APEC-02 to E. coli HS-4. All eight SCFAs significantly reduced transconjugant populations (FIG. 4).

### Example 2: SCFAs in Ex Vivo Bacterial Conjugation in Chicken Ceca Explants

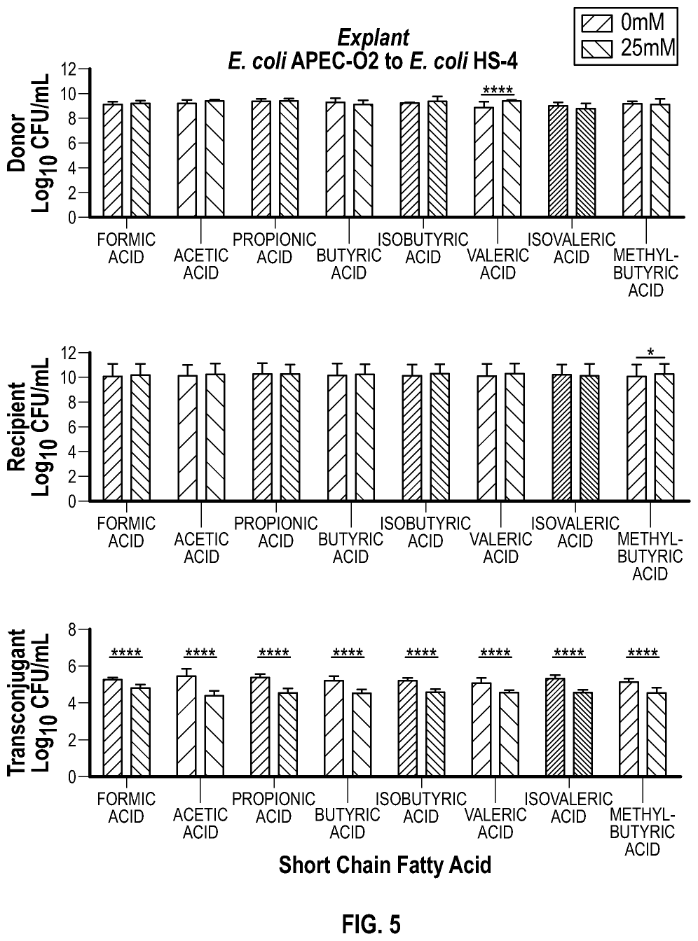

Ex vivo ceca explants serve as models for bacterial host interactions. Plasmid transfer and its inhibition by SCFAs were tested using chicken ceca explants to mimic in vivo conditions. Chicken ceca explants were harvested from 2 week-old chickens, cultured in vitro, and used in co-culture with donors/recipients in the presence or absence of SCFAs. Eight distinct SCFAs (formic acid, acetic acid, propionic acid, butyric acid, isobutyric acid, valeric acid, isovaleric acid, methylbutyric acid) at 25 mM were added with the conjugation pair E. coli APEC-02 to E. coli HS-4. All eight SCFAs showed efficacy when used individually (FIG. 5).

### Example 3: Prebiotics Fermented into SCFAs

Free SCFAs are rapidly absorbed in the small intestine. Prebiotics, instead, escape digestion in the small intestine and are fermented into SCFAs in the ceca, thereby increasing SCFA concentrations. The in vivo role of prebiotic-derived SCFAs on the inhibition of HGT and AR incidence both in the naïve and challenged gut microbiotas will be examined. Following the guidance of Iowa State IACUC protocol, day-old White-Leghorn chickens will be acclimated 3 days and given standard feed (Purina organic chicken starter) and water ad libitum. Chickens will be fed 0 or 0.5% mannan-, galacto-, or fructo-oligosaccharide (MOS, GOS, FOS) prebiotics. Half of each treatment group will be orally inoculated with plasmid donor S. Kentucky CVM29188, and resident E. coli will act as a recipient. Fecal populations of the donor, resident Enterobacteriaceae recipients, and transconjugants will be enumerated on selective media. Ceca contents will be analyzed by gas chromatography for SCFA concentrations and shotgun metagenomic sequencing to evaluate the microbiota and resistome. Fecal contents will be collected for in vitro conjugation inhibition assays. Plasmid transfer is expected to be reduced in hosts with elevated cecal concentrations of SCFAs in the prebiotics-added diet group compared to the control (no-prebiotics).

### Example 4: Inhibition of Bacterial Conjugation of Various Plasmid Incompatibility Types

A single bacterial strain, Escherichia coli strain SP915 (MG1655 derivative) was used as the bacterial plasmid donor for the three plasmids pKJK5-GM (IncP), pCVM29188_146 (IncF) and pC20-GM (IncI). Plasmid transfer was assessed in conjugation containing the donor and the recipient Escherichia coli strain HS-4. Cultures were grown overnight and standardized to an optical density of approximately 1.0 at 600 nm in fresh Luria broth media, and mixed 1:1 and incubated with the addition of either sterile distilled water, or 0.025 M short chain fatty acid (acetate, propionate, or butyrate) for six hours at 37° C. Solutions were serially diluted and plated on selective media for enumeration.

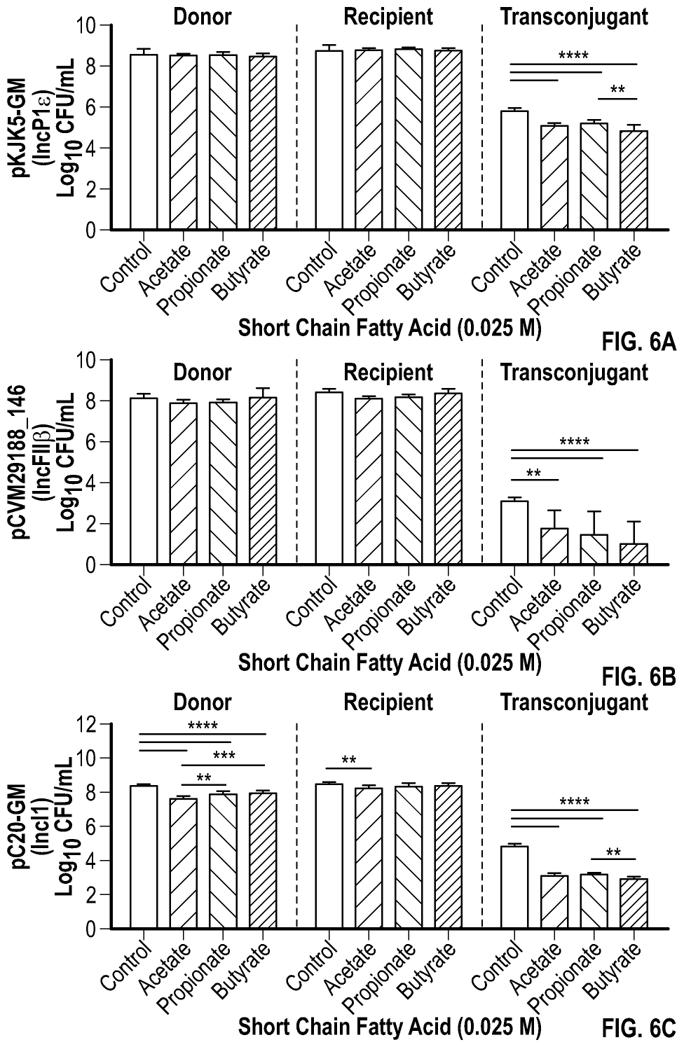

A significant reduction in Logic) transformed colony forming units per 1 mL volume of transconjugants (recipients that acquire the plasmid) between SCFA treated groups compared to water treated controls was observed in all three plasmids tested (FIG. 6). These data demonstrate there is a broad ability of these treatments to impact bacterial plasmid transfer which may occur in the gut environment. SOFA treatments inhibit the transfer of not only the narrow host range IncF plasmid incompatibility group, but also the broad host range IncP and narrow host range IncI plasmid incompatibilities group.

What is claimed is:

1. A method of inhibiting bacterial conjugation or horizontal gene transfer in the gut of a subject, the method comprising:
administering to the subject a composition comprising a probiotic bacterium and a prebiotic, wherein the probiotic bacterium produces at least one short chain fatty acid in the gut of the subject.

2. The method of claim 1, wherein the probiotic bacterium comprises a Lactobacillus spp.

3. The method of claim 1, wherein the prebiotic comprises fructo-oligosaccharides, galacto-oligosaccharides, mannan-oligosaccharides, or a combination thereof.

4. The method of claim 1, wherein the short chain fatty acid comprises formic acid, acetic acid, propionic acid, butyric acid, isobutyric acid, valeric acid, isovaleric acid, methylbutyric acid, or a combination thereof.

5. The method of claim 1, wherein the composition further comprises a carrier.

6. The method of claim 1, wherein the composition is administered orally.

7. The method of claim 1, wherein the composition is administered with food or feed.

8. The method of claim 1, wherein the subject is a poultry animal, a pig, a cow, or a human.

9. A method of inhibiting bacterial conjugation or horizontal gene transfer in the gut of a subject, the method comprising:
administering to the subject a composition comprising at least one short chain fatty acid.

10. The method of claim 9, wherein the short chain fatty acid comprises formic acid, acetic acid, propionic acid, butyric acid, isobutyric acid, valeric acid, isovaleric acid, methylbutyric acid, or a combination thereof.

11. The method of claim 9, wherein the composition further comprises a carrier.

12. The method of claim 9, wherein the composition is administered orally.

13. The method of claim 9, wherein the composition is administered with food or feed.

14. The method of claim 9, wherein the subject is a poultry animal, a pig, a cow, or a human.

15. A composition for inhibiting bacterial conjugation or horizontal gene transfer in the gut of a subject comprising at least one short chain fatty acid and/or a short chain fatty acid-producing probiotic bacterium.

16. The composition of claim 15, further comprising a prebiotic, wherein the prebiotic comprises fructo-oligosaccharides, galacto-oligosaccharides, mannan-oligosaccharides, or a combination thereof.

17. The composition of claim 15, wherein the probiotic bacterium comprises a Lactobacillus spp.

18. The composition of claim 15, wherein the composition further comprises a carrier.

19. The composition of claim 15, wherein the composition is formulated for oral administration.

20. The composition of claim 15, wherein the composition is a food or feed composition.
